(19) United States
(12) Patent Application Publication
(10) Pub. No.: US 2014/0273045 A1
(43) Pub. Date: Sep. 18, 2014

(54) Modular Biochemical Signaling Laboratory Breadboard for Disease Research, Drug Discovery, Cell Biology, and Other Applications

(71) Applicant: Lester F. Ludwig, San Antonio, TX (US)

(72) Inventor: Lester F. Ludwig, San Antonio, TX (US)

(21) Appl. No.: 14/216,420

(22) Filed: Mar. 17, 2014

Related U.S. Application Data
(60) Provisional application No. 61/802,127, filed on Mar. 15, 2013.

(51) Int. Cl. C12Q 1/48

(52) U.S. Cl. CPC C12Q 1/485
USPC 435/15

(57) ABSTRACT

A “breadboard” approach by which a biochemical signaling process, pathway, or network under study is separated or segmented into interconnected smaller portions, at least one of which can to a degree of approximation be accurately emulated with a replica microscale and/or nanoscale fluidic implementation whose constituent species can be closely controlled and at least one aspect of whose behavior can be measured. Control and measurement information interfaces with a computer that executes algorithms comprising one or more of a control process, control event-script, experiment, data recording, and mathematical model. A model can be used to simulate the actions, behavior, or other aspects of another portion of the biochemical signaling process, pathway, or network. Replica constituents can include enzymes, other proteins, lipids, ions, peptides, and other materials provided under controlled conditions and timing, as well as varying degrees of competitive species, drugs, environmental influences, and substitute or representative molecular crowding.

### CROSS-REFERENCE TO RELATED APPLICATIONS

This application claims benefit of priority from Provisional U.S. Patent application Ser. No. 61/802,127, filed Mar. 15, 2013, the contents of which are incorporated by reference.

### COPYRIGHT & TRADEMARK NOTICES

A portion of the disclosure of this patent document may contain material, which is subject to copyright protection. Certain marks referenced herein may be common law or registered trademarks of the applicant, the assignee or third parties affiliated or unaffiliated with the applicant or the assignee. Use of these marks is for providing an enabling disclosure by way of example and shall not be construed to exclusively limit the scope of the disclosed subject matter to material associated with such marks.

### BACKGROUND

1. Field of the Invention

The present disclosure pertains to next generation tools for the study of biological signaling processes and networks in living biological cells, and in particular relates to laboratory tools, methods, microscale sensors, microscale instrumentation, microfluidic, computerized instrumentation, computer simulation, and computer analysis tools relating to the study, analysis, and modeling of biological cell signaling. Aspects of the present application can also be readily used or adapted for the study, analysis, and modeling of biochemical processes and pathways for metabolism and gene regulation as well as laboratory or industrial activities pertaining to confined-environment chemistry, intercalation chemistry, chemical reactions in constrained systems, molecular encapsulation, and aspects of host-guest chemistry and the synthesis and study of meta-materials.

2. Related Art

Material related to the topic of this patent application is provided in two earlier pending patent applications by the present inventor, specifically U.S. patent application Ser. No. 13/157,304 and paragraphs [00565] through [00569] of U.S. patent application Ser. No. 13/761,142. Pending patent application U.S. Ser. No. 13/761,142 additionally teaches throughout a variety of microscale sensors, microscale instrumentation, computer-controlled microfluidics, and computer-controlled instrumentation systems and methods useful to the present patent application. U.S. Pat. No. 8,594,848, also by the present inventor, teaches throughout additional computer-controlled microfluidics, microscale instrumentation, and computer-controlled instrumentation systems and methods useful to the present patent application. Allowed patent application U.S. Ser. No. 12/931,867, also by the present inventor, teaches throughout various microfluidic chemistry and further computer-controlled chemical processing systems and methods useful to the present patent application. U.S. Pat. Nos. 8,032,258 and 8,606,414 and pending U.S. patent application Ser. No. 13/251,288, also by the present inventor, teach controllable multichannel microfluidic chemical bus systems and methods useful to the present patent application.

### SUMMARY

The present application is directed to creation of a new type of tool for research in the areas of biochemical signaling, disease processes, drug discovery, cell biology, and other applications.

For purposes of summarizing, certain aspects, advantages, and novel features are described herein. Not all such advantages may be achieved in accordance with any one particular embodiment. Thus, the disclosed subject matter may be embodied or carried out in a manner that achieves or optimizes one advantage or group of advantages without achieving all advantages as may be taught or suggested herein.

The approach adapts the concept of a “breadboard” such as the electronic breadboards used in electronic circuitry prototyping and optical breadboards used in optical system R&D.

As to this, the present disclosure includes approaches for the selective piecewise construction of replicas of portions of naturally-occurring biochemical processes and pathways for signaling, metabolism, and gene regulation. These replicas can for example be implemented in microscale and nanoscale fluidic environments, can internally comprise one or more microscale and nanoscale fluidic environments, can be computer-controlled, and can comprise extensive monitoring via internal sensors, external sensors, and other types of instrumentation. The replicas can for example additionally be arranged to include a variety of constituent species such as enzymes, other proteins, lipids, ions, peptides, and other materials, and the introduction of such constituent species can be provided under controlled conditions under controlled timing. The replicas can for example additionally be arranged to include the controlled presence and controlled introduction of varying degrees of competitive species, drugs, and environmental influences (hormone, temperature, chemical, etc.). Further, the replicas can be arranged to include controlled degrees of substitute or representative molecular crowding.

The embodiments of the present application are directed to address problems in the study, analysis, and modeling of biological signaling processes and networks in living biological cells. Because of this and the “breadboard” abstraction adopted from electronics prototyping and optical R&D, embodiments of the present application will be referred to as a “biological signaling breadboard” for convenience. Despite that naming, embodiments of the present application can be readily used or adapted for the study, analysis, and modeling of biochemical processes and pathways for metabolism and gene regulation as well as laboratory or industrial activities pertaining to confined-environment chemistry, intercalation chemistry, chemical reactions in constrained systems, molecular encapsulation, and aspects of host-guest chemistry and the synthesis and study of meta-materials.

Embodiments of the biological signaling breadboard or features therein can comprise one or more microscale or nanoscale chemical reaction environments, each for example chemical reaction environment arranged to:
accept reactants, reagents, and other material,
comprise at least one reaction environment,
include or support sensors or internal instrumentation for monitoring one or more of:
   the presence or concentration of chemical/biochemical species,
   the presence and progress of chemical/biochemical processes,
include or support aspects of external instrumentation for monitoring one or more of:
   the presence or concentration of chemical/biochemical species,
   the presence and progress of chemical/biochemical processes,
provide controlled introduction of one or more chemical/biochemical materials,
provide controlled stimulus to initiate or maintain one or more chemical/biochemical processes.

Embodiments of the biochemical signaling breadboard can further be configured to interface with a computing system performing one or more of the following functions:
Receive measurement information from the sensors and/or instrumentation associated with each of the one or more microscale or nanoscale chemical reaction environments;
Transmit control information used to control fluidics systems.
Transmit control information used to control the introduction of one or more chemical/biochemical materials into each of the one or more microscale or nanoscale chemical reaction environments;
Transmit control information used to control the stimulus of one or more chemical/biochemical processes into each of the one or more microscale or nanoscale chemical reaction environments;
Execute control algorithms for creating and timing the transmitting of the aforementioned control information;
Execute feedback control algorithms for creating and timing the transmitting of the aforementioned control information responsive to received measurement information;
Execute storage algorithms for at least storing the aforementioned measurement information to create stored measurement information;
Execute retrieval algorithms for at least retrieving the aforementioned stored measurement information;
Execute control algorithms for creating and timing the transmitting of the aforementioned control information responsive to retrieved stored measurement information;
Execute analysis algorithms for at least analyzing the aforementioned measurement information;
Execute storage algorithms for at least storing the aforementioned analysis information to create stored analysis information;
Execute retrieval algorithms for at least retrieving the aforementioned stored analysis information;
Execute control algorithms for creating and timing the transmitting of the aforementioned control information responsive to retrieved stored analysis information;
Support the use of scripts and script-driven control algorithms;
Provide user interface functions.
   In some embodiments the computing system also is executing a mathematical model, for example as part of the aforementioned algorithms or in communication with the aforementioned algorithms.

In embodiments where there are more than one microscale or nanoscale chemical reaction environments, where advantageous, two or more of these microscale or nanoscale chemical reaction environments can be configured to comprise linking algorithms or other arrangements wherein received measurement information associated with one of the microscale or nanoscale chemical reaction environments is used for creating and timing the transmitting of control information directed to at least one other of the microscale or nanoscale chemical reaction environments.

In embodiments comprising at least one such linking algorithm, the combination of controlled and (sensor or instrument) monitored microscale or nanoscale chemical reaction environments together with the linking algorithms can be used to emulate a signaling cascade or portion of a signaling network.

In embodiments comprising a plurality of such linking algorithm, the combination of controlled and (sensor or instrument) monitored microscale or nanoscale chemical reaction environments together with the linking algorithms can be used to emulate a larger signaling cascade, portion of a signaling network, or entire signaling network.

In embodiments comprising a plurality of such linking algorithms, and wherein the linking introduces a feedback loop involving two or more of the microscale or nanoscale chemical reaction environments, the combination of controlled and (sensor or instrument) monitored microscale or nanoscale chemical reaction environments together with the linking algorithms can be used to emulate signaling feedback, a signaling cascade with feedback, a portion of a signaling network comprising feedback, or entire signaling network comprising feedback.

In embodiments comprising a plurality of such linking algorithms, and wherein the linking introduces a feedforward path involving two or more of the microscale or nanoscale chemical reaction environments, the combination of controlled and (sensor or instrument) monitored microscale or nanoscale chemical reaction environments together with the linking algorithms can be used to emulate signaling feedforward, a signaling cascade with feedforward, a portion of a signaling network comprising feedforward, or entire signaling network comprising feedforward paths.

Embodiments of the biochemical signaling breadboard can further be configured to include at least one fluidic interconnection between at least two of the microscale or nanoscale chemical reaction environments. In some embodiments the fluidic interconnection is simply gated on and off under computer control. In other embodiments, the fluidic interconnection is realized as one possible configuration of a computer-controlled reconfigurable fluidic interconnection network. In some embodiments the computer-controlled reconfigurable fluidic interconnection network can be implemented as or comprise aspects of a controllable multichannel microfluidic chemical bus such as that taught in pending U.S. Pat. Nos. 8,032,258 and 8,606,414 and pending U.S. patent application Ser. No. 13/251,288.

In embodiments comprising at least one such fluidic interconnection, the combination of controlled and (sensor or instrument) monitored microscale or nanoscale chemical reaction environments together with the linking algorithms can be used to emulate a signaling cascade or portion of a signaling network.

In embodiments comprising a plurality of such fluidic interconnections, the combination of controlled and (sensor or instrument) monitored microscale or nanoscale chemical reaction environments together with the linking algorithms can be used to emulate a larger signaling cascade, portion of a signaling network, or entire signaling network.

In embodiments comprising a plurality of such fluidic interconnections, and wherein the linking introduces a feedback loop involving two or more of the microscale or nanoscale chemical reaction environments, the combination of controlled and (sensor or instrument) monitored microscale or nanoscale chemical reaction environments together with the linking algorithms can be used to emulate signaling feedback, a signaling cascade with feedback, a portion of a signaling network comprising feedback, or entire signaling network comprising feedback.

In embodiments comprising a plurality of such fluidic interconnections, and wherein the linking introduces a feedforward path involving two or more of the microscale or nanoscale chemical reaction environments, the combination of controlled and (sensor or instrument) monitored microscale or nanoscale chemical reaction environments together with the linking algorithms can be used to emulate signaling feedforward, a signaling cascade with feedforward, a portion of a signaling network comprising feedforward, or entire signaling network comprising feedforward paths.

### BRIEF DESCRIPTION OF THE DRAWINGS

The above and other aspects, features and/or advantages of the present application may become more apparent upon consideration of the following description of embodiments taken in conjunction with the accompanying drawing figures, wherein:

FIG. 1a illustrates a spectrum of tools used in the study of biochemical signaling networks.

FIG. 1b illustrates a relative role of an embodiment of the present application in the spectrum depicted in FIG. 1a.

FIG. 2 illustrates a representation of recent understanding of the EGF (Epidermal Growth Factor) induced MAP (Mitogen-Activated Protein) Kinase Signal Transduction Pathway.

FIG. 3 illustrates an example representative pathway segment such as that found in biochemical signaling pathways.

FIG. 4a illustrates an example partition of the example representative pathway segment depicted in FIG. 3.

FIG. 4b illustrates another example partition of the example representative pathway segment depicted in FIG. 3.

FIG. 4c illustrates the entire example representative pathway segment depicted in FIG. 3.

FIG. 5a illustrates an example embodiment comprising a plurality of computer-controlled and computer-monitored microscale or nanoscale chemical reaction environments and a computer-controlled arrangement for executing algorithms and interfacing with the plurality of computer-controlled and computer-monitored microscale or nanoscale chemical reaction environments.

FIG. 5b illustrates an example variation on the example embodiment depicted in FIG. 5a that additionally incorporates controlled fluidic interconnection among at least two of the microscale or nanoscale chemical reaction environments.

FIG. 6 illustrates an example approach to emulating molecular crowding through the controlled introduction of molecular crowding constituents into a monitored microscale or nanoscale chemical reaction environment.

FIG. 7a illustrates an example variation on the example embodiment depicted in FIG. 5a that incorporates the approach to emulating molecular crowding depicted in FIG. 6.

FIG. 7b illustrates an example variation on the example embodiment depicted in FIG. 7a.

FIG. 8 illustrates an example variation on the approach to emulating molecular crowding depicted in FIG. 6.

FIG. 9a illustrates an example variation on the example embodiment depicted in FIG. 7a that incorporates the approach to the dispensing of drug constituents depicted in FIG. 8.

FIG. 9b illustrates an example variation on the example embodiment depicted in FIG. 9a.

### DETAILED DESCRIPTION

In the following description, reference is made to the accompanying drawing figures which form a part hereof, and which show by way of illustration specific embodiments of the present application. It is to be understood by those of ordinary skill in this technological field that other embodiments may be utilized, and structural, electrical, as well as procedural changes may be made without departing from the scope of the present application.

In the following description, numerous specific details are set forth to provide a thorough description of various embodiments. Certain embodiments may be practiced without these specific details or with some variations in detail. In some instances, certain features are described in less detail so as not to obscure other aspects. The level of detail associated with each of the elements or features should not be construed to qualify the novelty or importance of one feature over the others.

Biochemical signaling networks play considerable roles in the cell cycle and most diseases, for example cancer. In addition to complicated multiple feedback loops and feedforward paths that regulate resulting dynamics, biochemical signaling networks include crosstalk among pathways, mechanical aspects of transport, conformation-dependent allosteric (state-dependent) reaction dynamics, enzyme recovery dynamics, exogenous regulatory controls, and many other exotic processes that are both critical to life processes and extremely complex. These render complex dynamics whose normal behavior, pathologies, and sensitivities are barely understood.

A more extensive analytical, quantitative, confirmative, and predictive understanding of biochemical signaling process, pathways, and networks is becoming increasingly indispensable. Comprehensive, accurate understanding and predictive modeling of biochemical signaling will play critical roles in future diagnostics and drug discovery, increasing replacing the roles of QSAR and other expensive and extensive approaches which, after a spectacular run of initial valuable productivity, have entirely failed to deliver new drugs. Further, the wide variability of side effects (both traceable and not traceable to the presence of undesirable enantiomers resultant from drug manufacturing processes) depends on the variability among patients' personal metabolic and signaling makeup. Additionally, many diseases such as cancer (lung cancer being a vibrant example) appear mechanistically to be optimally conquered via highly individualized analysis and therapies not unlike the notions of personalized or individualized medicine that are advocated for other sometimes controversial reasons. At the structural center of realistic approaches to all of these is again a comprehensive, accurate understanding and predictive modeling of biochemical signaling processes and signaling networks.

To date the approaches and results are at once both spectacular and primitively crude. A large number of signaling processes and pathways have been identified, with many new ones identified or conjectured every month. Many of these newly identified or conjectured signaling processes and pathways provide brand new understandings and explanations, and at times entirely unknown new phenomena. Experimental study of signaling processes and pathways have employed an impressive spectrum of technologies and methods but in many ways are limited to the introduction of instrumentation-observable markers into living cells, the testing of gross-effect biomarkers produced by living cells, laboratory-scale biochemical reaction studies, and probing of large molecule structure by biophysics and spectroscopic techniques. As powerful as these techniques have proven to be, they still suffer from immense limitation with regards to the types of behavior they can observe and characterize.

The scale, nonlinearities, and interconnected complexity of biochemical signaling networks have been initially addressed with attempts to modularize. A common approach is the partition of complex signaling networks into small sections that are characterized as behaving like combinational and state-retaining logic circuits, i.e., so-called signaling “motifs.” However, the scale, interconnected complexity, and adaptively of biochemical signaling networks exceeds human comprehension and defies attempts to modularize. In many cases, conceptual modeling of biological signaling network has proven ineffective and at time deceptive as it is mentally impossible to juggle large pathways involving many components and because the mathematical behavior is too hard to intuitively characterize. In many ways the predictive modeling of signaling networks has exceeded what can be done without computer support.

Further, the explosion of genomic and proteomic laboratory analysis, bioinformatics, research publications, and “big data” analysis has created vast tomes of unverified signaling network models. Many of these are the result of automated statistical analysis of the results of automatic inferences drawn from automatic word searches on research publications text, be they speculative, unconfirmed, or robust verified. To the extent that there is some valid degree of topological accuracy, reaction rates and many other parameters required for accurate analytical modeling are often unavailable. Further, the measurements of reaction rates and other parameters required for accurate analytical modeling is often made under artificial and inaccurate circumstances, for example not including the profound effects of molecular crowding, localized and confined reaction environments, or even being able to accurately control for other potentially interfering processes.

Summarizing some of the points thus far together with some additional remarks:
The long celebrated and indoctrinated methods of drug design and testing are failing to produce and producing exploding costs;
For many reasons the best candidates for the next step is the leveraging of analytical, quantitative, confirmative, and predictive understanding of biochemical signaling process, pathways, and networks;
For this and yet other reasons, an extensive analytical, quantitative, confirmative, and predictive understanding of biochemical signaling process, pathways, and networks is becoming increasingly indispensable for drug design, therapies, treatment of disease, and the control of destructive side effects;
The scale, nonlinearities, interconnected complexity, and adaptively of biochemical signaling networks exceeds human comprehension and defies attempts to modularize;
The need for accurate large-scale computer models of biochemical networks is urgent as there is vast need for predictive modeling of signaling networks, yet the predictive modeling has exceeded what can be done without computer support;
However, the powerful tools and methods employed to date for characterizing signaling processes, signaling network topologies, and signaling process modeling parameters are still primitive and subject to immense error.

As to some of the powerful tools and methods employed to date for characterizing biochemical signaling processes, signaling network topologies, and signaling process modeling parameters, FIG. 1a depicts an example spectrum of tools used in the study of biochemical signaling networks. Not explicitly depicted is the combined use of genomic and proteomic laboratory analysis, bioinformatics, research publications, and “big data” analysis in the creation of huge unverified signaling network models from automated statistical analysis of the results of automatic inferences drawn from automatic word searches on research publications text, be they speculative, unconfirmed, or robust verified; if these are to be included for some sense of completeness (as it is arguably a huge area of recent activity) these could fit in the meeting ground between the depicted group categories of “physical” and “mathematical” methodologies. The intended point of FIG. 1a, however, is that there is a spectrum of methodologies ranging from attempts to directly and accurately observe biochemical signaling processes, signaling network topologies, and signaling process modeling parameters on one extreme to attempts to directly and accurately model with abstract mathematical models that hold the promise of providing predictive analytical and design tools.

In mechanical, electrical, optical, chemical, material science, and other forms of engineering there has been great success in creating abstract mathematical models (“CAD tools”) that accurate provide predictive analytical and design tools. These tools incorporate vast amounts of confirmed physical science, accurate physical measurements, and confirmed analytical models combined into a comprehensive framework, and virtually no contemporary suspension bridge, transportation vehicle, integrated circuit, consumer product, or chemical plant is designed without such tools. However, such tools have not yet become possible because of the shortcomings and shortages of needed confirmed physical science, needed accurate physical measurements, needed confirmed analytical models, and needed comprehensive framework.

Each of the five examples cited in the example spectrum of tools used in the study of biochemical signaling networks shown in FIG. 1a involves a number of limitations as described earlier. For example, the physical measurements of reaction rates and other parameters required for accurate analytical modeling is often made under artificial and inaccurate circumstances, for example not including the profound effects of molecular crowding, localized and confined reaction environments, or even being able to accurately control for other potentially interfering processes. On the other side of the spectrum, computer simulations and analytical models suffer from, among other things, the lack of accurate physical measurements, confirmed accurate analytical models, adequate level of scale, accurate signaling network topologies, and overall comprehensive framework.

The present disclosure describes approaches to additional tools for the study of biological cell signaling employing methods, microscale sensors, microscale instrumentation, microfluidic, and computerized instrumentation. The approach adapts the concept of a “breadboard” such as the electronic breadboards used in electronic circuitry prototyping and optical breadboards used in optical system R&D. Embodiments of the present application will be referred to as “biological signaling breadboards” for convenience. Results from and the environments comprised by embodiments of the present application can be used by and combined with computer simulation and computer analysis tools relating to the study, analysis, and modeling of biological cell signaling, for example the modeling environment described in U.S. Pat. No. 8,660,823 by the present inventor. For example, results from and the environments comprised by embodiments of the present application can be used by and combined with computer simulation and computer analysis tools in manners such as those described in pending U.S. patent application Ser. No. 13/157,304.

Among other things, the present disclosure describes approaches for selective piecewise construction of replicas of portions of naturally-occurring biochemical processes and pathways for signaling, metabolism, and gene regulation. These replicas can for example be implemented in microscale and nanoscale fluidic environments, can internally comprise one or more microscale and nanoscale fluidic environments, can be computer-controlled, and can comprise extensive monitoring via internal sensors, external sensors, and other types of instrumentation. The replicas can for example additionally be arranged to include a variety of constituent species such as enzymes, other proteins, lipids, ions, peptides, and other materials, and the introduction of such constituent species can be provided under controlled conditions under controlled timing. The replicas can for example additionally be arranged to include the controlled presence and controlled introduction of varying degrees of competitive species, drugs, and environmental influences (hormone, temperature, chemical, etc.). Further, the replicas can be arranged to include controlled degrees of substitute or representative molecular crowding.

In embodiments where there are more than one microscale or nanoscale chemical reaction environments, where advantageous, two or more of these microscale or nanoscale chemical reaction environments can be configured to comprise linking algorithms or other arrangements wherein received measurement information associated with one of the microscale or nanoscale chemical reaction environments is used for creating and timing the transmitting of control information directed to at least one other of the microscale or nanoscale chemical reaction environments.

The embodiments of the present application are directed to the study, analysis, and modeling of biological cell signaling, and also could be used for the study, analysis, and modeling of biochemical processes and pathways for metabolism and gene regulation. In that it can combine physical measurements with computer control driving by mathematical models, FIG. 1b depicts an example relative role of an embodiment of the present application straddling “physical” and “mathematical” methodologies in the spectrum depicted in FIG. 1a.

Additionally, aspects of the present application can also be readily used or adapted for laboratory or industrial activities pertaining to confined-environment chemistry, intercalation chemistry, chemical reactions in constrained systems, molecular encapsulation, and aspects of host-guest chemistry and the synthesis and study of meta-materials.

Naturally-Occurring Biochemical Signaling Pathways

As described above, network topology graphs (the term “graph” here being the mathematical term meaning a collection of nodes and a collection of links or “edges” interconnecting these nodes) for naturally-occurring biochemical signaling pathways can be quite large and are always subject to change subject to new findings and their acceptance. As a representative example, FIG. 2 depicts a representation of recent understanding of the EGF (Epidermal Growth Factor) induced MAP (Mitogen-Activated Protein) Kinase Signal Transduction Pathway, one of the most important pathways in mammalian cells for regulating cell growth, survival, proliferation, and differentiation. Accordingly, this is one of the most experimentally and computationally investigated cellular signaling pathways, with numerous dynamic analysis and computational models available in the literature. (A popular review article on this pathway providing extensive citations is that of Oda, Matsuoka, Funahashi, Kiano “A Comprehensive Pathway Map of Epidermal Growth Factor Receptor Signaling,” Molecular Systems Biology, 1:2005.0010, May 25, 2005.)

This pathway could be separated or segmented into interconnected smaller portions of the overall pathway with, for example, pair-wise interfaces among smaller portions of the overall pathway, these pair-wise interfaces implementing or supporting the interconnection. Leveraging this general approach provides a basis for creating a “biological signaling breadboard” as will be described. A larger pathway can be separated or segmented into interconnected smaller portions, at least one of which can be to some degree of controlled approximation be accurately emulated with some form of replica microscale and/or nanoscale fluidic implementation whose constituent species can be closely controlled and at least one aspect of whose biochemical behavior can be closely measured by some means with adequate accuracy. The control and measurement information can be interfaced with a computer that executes algorithms comprising for example one or more of a control process, control event-script, experiment, data recording, and mathematical model.

Example Partitions of Natural Biochemical Signaling Pathways

As an example, FIG. 3 depicts a representative pathway segment such as that found in biochemical signaling pathways. Herein, an incoming stimulus from external source X activates B, which in turn activates C; the activations of C is both passed on to external respondent Z as well as internal respondent D, the latter which in turn activates E subject to inhibition by external source Y. The activation of E in turn inhibits the aforementioned pathway whereby B is activated by A.

FIG. 4a depicts an example partition of the example representative pathway segment depicted in FIG. 3, wherein a first partition comprises A, B, the pathway from A to B, and an inhibitor input to that pathway from A to B, a second partition comprises D, E, the pathway from D to E, and an inhibitor input to the pathway from D to E. The partition excludes C.

FIG. 4b depicts another example partition of the example representative pathway segment depicted in FIG. 3, wherein a first partition comprises A, B, C, the pathway between A and B, an inhibitor input to the pathway from A to B, and the pathway from B to C.

FIG. 4c depicts the entire example representative pathway segment depicted in FIG. 3, so the partition is simply the separation for X, Y, and Z. In an embodiment, the entire example representative pathway segment depicted in FIG. 3 is implemented with computer-controlled and computer-monitored microscale or nanoscale chemical reaction environment linked to at least one algorithm executing on a computer, and the roles of X, Y, and Z are handled by at least one algorithm executing on the computer or a related computer.

Implementing Replica Partition Chemical Reaction Environments

Embodiments of the biological signaling breadboard or features therein can comprise one or more microscale or nanoscale chemical reaction environments that can be used to implement a replica partitioned portion of larger biochemical processes and pathways for signaling, metabolism, and gene regulation. For example, such replica partitioned chemical reaction environments can be arranged to:
accept reactants, reagents, and other material passively and/or responsive to computer control via fluidic inputs,
comprise at least one reaction environment,
include or support sensors or internal instrumentation for monitoring one or more of:
   the presence or concentration of chemical/biochemical species,
   the presence and progress of chemical/biochemical processes,
include or support aspects of external instrumentation for monitoring one or more of:
   the presence or concentration of chemical/biochemical species,
   the presence and progress of chemical/biochemical processes,
provide controlled introduction of one or more chemical/biochemical materials,
provide a controlled environment required maintain one or more chemical/biochemical processes,
provide controlled stimulus to initiate or maintain one or more chemical/biochemical processes.

In many embodiments, replica partitioned chemical reaction environments can be arranged to provide outlets for removing the reaction products.

In some embodiments, replica partitioned chemical reaction environments can comprise at least one membrane.

Functionally Interconnecting Replica Partition Chemical Reaction Environments with a Computer and Algorithms for Chemical Reaction Environment Control and Chemical Reaction Environment Measurements

FIG. 5a depicts an example embodiment comprising a plurality of computer-controlled and computer-monitored microscale or nanoscale chemical reaction environments and a computer-controlled arrangement for executing algorithms and interfacing with the plurality of computer-controlled and computer-monitored microscale or nanoscale chemical reaction environments.

FIG. 5b depicts an example variation on the example embodiment depicted in FIG. 5a that additionally incorporates controlled fluidic interconnection among at least two of the microscale or nanoscale chemical reaction environments.

Embodiments of the biochemical signaling breadboard can further be configured to interface with a computing system performing one or more of the following functions:
Receive measurement information from the sensors and/or instrumentation associated with each of the one or more microscale or nanoscale chemical reaction environments;
Transmit control information used to control fluidics systems.
Transmit control information used to control the introduction of one or more chemical/biochemical materials into each of the one or more microscale or nanoscale chemical reaction environments;
Transmit control information used to control the stimulus of one or more chemical/biochemical processes into each of the one or more microscale or nanoscale chemical reaction environments;
Execute control algorithms for creating and timing the transmitting of the aforementioned control information;
Execute feedback control algorithms for creating and timing the transmitting of the aforementioned control information responsive to received measurement information;
Execute storage algorithms for at least storing the aforementioned measurement information to create stored measurement information;
Execute retrieval algorithms for at least retrieving the aforementioned stored measurement information;
Execute control algorithms for creating and timing the transmitting of the aforementioned control information responsive to retrieved stored measurement information;
Execute analysis algorithms for at least analyzing the aforementioned measurement information;
Execute storage algorithms for at least storing the aforementioned analysis information to create stored analysis information;
Execute retrieval algorithms for at least retrieving the aforementioned stored analysis information;
Execute script-driven control algorithms for creating and timing the transmitting of the aforementioned control information responsive to retrieved stored analysis information;
Support the use of scripts and script-driven control algorithms;
Provide user interface functions.

Functionally Interconnecting Replica Partition Chemical Reaction Environments Using Computer Algorithms Invoking Chemical Reaction Environment Control Responsive to Chemical Reaction Environment Measurements

In embodiments where there are more than one microscale or nanoscale chemical reaction environments can be configured to comprise linking algorithms or other arrangements wherein received measurement information associated with one of the microscale or nanoscale chemical reaction environments is used for creating and timing the transmitting of control information directed to at least one other of the microscale or nanoscale chemical reaction environments.

In embodiments comprising at least one such linking algorithm, the combination of controlled and (sensor or instrument) monitored microscale or nanoscale chemical reaction environments together with the linking algorithms can be used to emulate a signaling cascade or portion of a signaling network.

In embodiments comprising a plurality of such linking algorithm, the combination of controlled and (sensor or instrument) monitored microscale or nanoscale chemical reaction environments together with the linking algorithms can be used to emulate a larger signaling cascade, portion of a signaling network, or entire signaling network.

In embodiments comprising a plurality of such linking algorithms, and wherein the linking introduces a feedback loop involving two or more of the microscale or nanoscale chemical reaction environments, the combination of controlled and (sensor or instrument) monitored microscale or nanoscale chemical reaction environments together with the linking algorithms can be used to emulate signaling feedback, a signaling cascade with feedback, a portion of a signaling network comprising feedback, or entire signaling network comprising feedback.

In embodiments comprising a plurality of such linking algorithms, and wherein the linking introduces a feedforward path involving two or more of the microscale or nanoscale chemical reaction environments, the combination of controlled and (sensor or instrument) monitored microscale or nanoscale chemical reaction environments together with the linking algorithms can be used to emulate signaling feedforward, a signaling cascade with feedforward, a portion of a signaling network comprising feedforward, or entire signaling network comprising feedforward paths.

In an embodiment, the two partitions depicted in FIG. 4a are implemented with computer-controlled and computer-monitored microscale or nanoscale chemical reaction environments linked by at least one algorithm executing on a computer, and the roles of X, Y, Z, and C are handled by at least one algorithm executing on the computer or a related computer.

In an embodiment, the two partitions depicted in FIG. 4a are implemented with computer-controlled and computer-monitored microscale or nanoscale chemical reaction environments linked by at least one algorithm executing on a computer, and the roles of X, Y, and Z are handled by at least one algorithm executing on the computer or a related computer.

Fluidically Interconecting Replica Partition Chemical Reaction Environments

Embodiments of the biochemical signaling breadboard can further be configured to include at least one fluidic interconnection between at least two of the microscale or nanoscale chemical reaction environments. In some embodiments the fluidic interconnection is simply gated on and off under computer control. In other embodiments the fluidic interconnection is realized as one possible configuration of a computer-controlled reconfigurable fluidic interconnection network. In some embodiments the computer-controlled reconfigurable fluidic interconnection network can be implemented as, or comprise aspects of, a controllable multichannel microfluidic chemical bus such as that taught in pending U.S. Pat. Nos. 8,032,258 and 8,606,414 and pending U.S. patent application Ser. No. 13/251,288.

In embodiments comprising at least one such fluidic interconnection, the combination of controlled and (sensor or instrument) monitored microscale or nanoscale chemical reaction environments together with the linking algorithms can be used to emulate a signaling cascade or portion of a signaling network.

In embodiments comprising a plurality of such fluidic interconnection, the combination of controlled and (sensor or instrument) monitored microscale or nanoscale chemical reaction environments together with the linking algorithms can be used to emulate a larger signaling cascade, portion of a signaling network, or entire signaling network.

In embodiments comprising a plurality of such fluidic interconnections, and wherein the linking introduces a feedback loop involving two or more of the microscale or nanoscale chemical reaction environments, the combination of controlled and (sensor or instrument) monitored microscale or nanoscale chemical reaction environments together with the linking algorithms can be used to emulate signaling feedback, a signaling cascade with feedback, a portion of a signaling network comprising feedback, or entire signaling network comprising feedback.

In embodiments comprising a plurality of such fluidic interconnections, and wherein the linking introduces a feedforward path involving two or more of the microscale or nanoscale chemical reaction environments, the combination of controlled and (sensor or instrument) monitored microscale or nanoscale chemical reaction environments together with the linking algorithms can be used to emulate signaling feedforward, a signaling cascade with feedforward, a portion of a signaling network comprising feedforward, or entire signaling network comprising feedforward paths.

Anomalous Diffusion Processes Resulting from Molecular Crowding and Confined/Constrained Biochemical Reaction Environments

An important aspect in a faithfully rendered replica chemical reaction environment is provision for the almost universally ignored need for the inclusion of the effects if not accurate emulation molecular crowding. Formulations, models, simulations, and emulations that do not include accurate provisions for molecular crowding provide incorrect molecular-transport statistical thermodynamics and can completely omit reaction processes that naturally occur in living cells. For example, the statistical thermodynamics for molecular-transport without consideration of molecular crowding are those of classical Brownian motion, which in turn provides statistically reproducibility of time-observables, ensemble averaging, long-time convergences, the interchangeable equivalence of time-averaging and ensemble-averaging, and other ergodic and related properties. In contrast, the dense macromolecular environment inside living cells induces molecular crowding that dramatically shifts the statistical thermodynamics for molecular-transport to at least two gross types of “anomalous diffusion” processes that absolutely do not share the many singular privileges of classical Brownian motion. For these, time-averaged observables are not reproducible, time-translation invariance are not respected, molecular-transport is not regularized, diffusion is jumpier and slower, and diffusion exponents differing significantly from the value of 1 provided by classical Brownian motion result, with implications as to whether molecular interactions involving reaction barriers and restricted alignments have sufficient time to occur. Anomalous diffusion processes for molecules traveling throughout the crowded molecular environment of a living cell can be more accuracy modeled with the Continuous Time Random Walk (“CTRW”), a non-ergodic random process demonstrating excellent agreement with a wide range of measurement subjects. Lattice models are employed for modeling diffusion-limited bimolecular reactions where a small number of reactants diffuse in a crowded environment among a much larger number of inert particles; these provide confirming related results from a colloidal physical chemistry viewpoint. Anomalous diffusion processes for molecules confined within a smaller spatial region, for example in the telomeres of a chromosome, a monomer in a polymer chain, or a molecule embedded in a membrane, is the Fractional Brownian Motion (“FBM”) process. In contrast to classical Brownian motion, diffusion exponents for appropriate CTRM models have values of ˜0.7 while diffusion exponents for appropriate FMB models can have value of ˜0.3. Accessible well-written accounts describing these and other recent appreciations of molecular crowding can be found in the article by E. Barkai, Y. Garini, and R. Metzler entitled “Strange Kinetics of Single Molecules in Living Cells” published in Physics Today, Vol. 65 No. 8, August 2012, pp. 29-35 and the chapter by A. Minton and G. Rivas entitled “Biochemical Reactions in the Crowded and Confined Physiological Environment: Physical Chemistry Meets Synthetic Biology” in The Minimal Cell: The Biophysics of Cell Compartment and the Origin of Cell Functionality, P. Luisi and P. Stano (eds.) 2011, ISBN 9048199433, pp. 73-89.

In addition to diffusion effects that can dramatically affect whether molecular interactions involving reaction barriers and restricted alignments have sufficient time to occur, molecular crowding and confined or constrained biochemical reaction environments are also thought and increasingly found to have important effects on molecular conformation that has significant influence on enabling and inhibiting reactions and protein interactions. Some representative examples are described in the article by H. Dong, S. Qin, and H-X. Zhou entitled “Effects of Macromolecular Crowding on Protein Conformational Changes” published in PLoS Computational Biology 6(7), Jul. 1, 2010. Of special consideration as well are the effects of molecular crowding and confined or constrained biochemical reaction environments on protein folding (see for example D. Homouz, L. Stagg, P. Wittung-Stafshede, and M. Cheung, “Macromolecular Crowding Modulates Folding Mechanism of α/β Protein Apoflavodoxin,” Biophys J. 2009 January; 96(2):671-80. doi: 10.1016/j.bpj.2008.10.014) and disordered proteins (see for example E. Cino, M. Karttunen, and W-Y Choy, Effects of Molecular Crowding on the Dynamics of Intrinsically Disordered Proteins, PLoS Computational Biology, Nov. 26, 2012).

Controlled Molecular Crowding Emulation

Thus not only would a model or simulation that does not account for molecular crowding encounter accuracy shortcomings, but an emulated or other experimental environment for studying the existence of and rate constants for biochemical reactions that occur in cells which does not account for molecular crowding can be expected to have significant, if not profound, accuracy shortcomings.

Accordingly, the present application provides for emulating molecular crowding through the controlled introduction of molecular crowding constituents into a monitored microscale or nanoscale chemical reaction environment. Replica partition chemical reaction environments can be arranged to include controlled degrees of substitute or representative molecular crowding. For example, FIG. 6 depicts an example approach to emulating molecular crowding through the controlled introduction of molecular crowding constituents into a monitored microscale or nanoscale chemical reaction environment. As another example, FIG. 7a depicts an example variation on the example embodiment depicted in FIG. 5a that incorporates the approach to emulating molecular crowding depicted in FIG. 6. Similarly, FIG. 7b depicts an example variation on the example embodiment depicted in FIG. 7a that additionally incorporates controlled fluidic interconnection among at least two of the microscale or nanoscale chemical reaction environments.

Examples of experiments with various concentrations of molecular crowding constituents can be found, for example, in the review article by N. Chebotareva, B. Kurganov, and N. Livanova entitled “Biochemical Effects of Molecular Crowding” published in Biochemistry (Moscow), Vol. 69, No. 11, 2004, pp. 1239-1251, this in turn translated from Biokhimiya, Vol. 69, No. 11, 2004, pp. 1522-1536. The present application provides a rich computer-controlled environment for emulating varying degrees of naturally-occurring molecular crowding through the controlled introduction of substitute or representative molecular crowding constituents.

Confined and Constrained Reaction Environments for Replica Partition Chemical Reaction Environments

Similarly, not only would a model or simulation that does not account for confined and constrained reaction environments encounter accuracy shortcomings, but an emulated or other experimental environment for studying the existence of and rate constants for biochemical reactions that occur in cells which does not account for confined and constrained reaction environments can be expected to have significant, if not profound, accuracy shortcomings.

Aformentioned examples of confined and constrained reaction environments inside living cells include telomeres of a chromosome, a monomer in a polymer chain, or a molecule embedded in a membrane, but many other examples are comprised in cell organelles. As mentioned earlier, these diffusion processes (exhibiting FBM behavior and having diffusion exponent value of for example ˜0.3) differ profoundly from classical Brownian motion (having diffusion exponent value of 1) and also differ significantly from the types of anomalous diffusion processes for molecules traveling throughout the crowded molecular environment of a living cell (exhibiting CTRW behavior and having diffusion exponent value of for example ˜0.7 and other relevant statistical differences from FBM behavior). Additionally, confined and constrained reaction environments inside living cells are thought to influence molecular conformation affecting reactions and protein interactions and also on protein folding.

Accordingly, the present application provides for replica partition chemical reaction environments to internally comprise one or more confined or constrained reaction environments for emulation of processes occurring within confined reaction environments inside living cells.

Confined and constrained reaction environments can be applicably implemented in a wide variety of ways including but not restricted to structured polymers, graphene and graphite structures, pillared clays and other types of structured clays, structured synthetic zeolites, inclusion compounds and cavity-containing supra-molecular compounds (for example cyclodextrins, calixarenes, and other organic host lattices), clathrates, liposomes, and various types of self-assembled supra-molecular structures. The present application also provides for the use of ordinary and controlled micelles where practical, advantageous, and feasible.

Controlled Dispensing of Drug Constituents into Replica Partition Chemical Reaction Environments

The present application provides for the controlled dispensing of drug constituents into replica partition chemical reaction environments using the same general system architecture. For example, FIG. 8 depicts an example variation on the approach to emulating molecular crowding depicted in FIG. 6 that further incorporates an approach the dispensing of drug constituents. As another example, FIG. 9a depicts an example variation on the example embodiment depicted in FIG. 7a that incorporates the approach to the dispensing of drug constituents depicted in FIG. 8. Similarly, FIG. 9b depicts an example variation on the example embodiment depicted in FIG. 9a that additionally incorporates controlled fluidic interconnection among at least two of the microscale or nanoscale chemical reaction environments.

Example Measurement Implementations

The present application provides for replica partition chemical reaction environments to internally comprise one or more of monitoring via internal sensors, monitoring via external sensors, and monitoring via other types of instrumentation such a microscopes, NMR systems, lensless optical microscopy and/or optical tomography such as that taught in pending U.S. patent application Ser. Nos. 12/817,107 and 14/105,108 by the present inventor, as well as a wide variety of other types of instruments.

Sensing methods for internal sensors can be implemented in a wide variety of ways, for example including but not restricted to optical methods involving limited-influence fluorophore and chromophore probes on reactant molecules, bioFET sensing of reaction product concentrations, and other techniques.

The terms “certain embodiments”, “an embodiment”, “embodiment”, “embodiments”, “the embodiment”, “the embodiments”, “one or more embodiments”, “some embodiments”, and “one embodiment” mean one or more (but not all) embodiments unless expressly specified otherwise. The terms “including”, “comprising”, “having” and variations thereof mean “including but not limited to”, unless expressly specified otherwise. The enumerated listing of items does not imply that any or all of the items are mutually exclusive, unless expressly specified otherwise. The terms “a”, “an” and “the” mean “one or more”, unless expressly specified otherwise.

The foregoing description, for purpose of explanation, has been described with reference to specific embodiments. However, the illustrative discussions above are not intended to be exhaustive or to limit the present application to the precise forms disclosed. Many modifications and variations are possible in view of the above teachings. The embodiments were chosen and described in order to best explain the principles of the present application and its practical applications, to thereby enable others skilled in the art to best utilize embodiments of the present application and various embodiments with various modifications as are suited to the particular use contemplated.

While aspects of the present application has been described in detail with reference to disclosed embodiments, various modifications within the scope of the present application will be apparent to those of ordinary skill in this technological field. It is to be appreciated that features described with respect to one embodiment typically can be applied to other embodiments.

The present application can be embodied in other specific forms without departing from the spirit or essential characteristics thereof. The present embodiments are therefore to be considered in all respects as illustrative and not restrictive, the scope of the present application being indicated by the appended claims rather than by the foregoing description, and all changes which come within the meaning and range of equivalency of the claims are therefore intended to be embraced therein.

Although exemplary embodiments have been provided in detail, various changes, substitutions and alternations could be made thereto without departing from spirit and scope of the disclosed subject matter as defined by the appended claims. Variations described for the embodiments may be realized in any combination desirable for each particular application. Thus particular limitations and embodiment enhancements described herein, which may have particular advantages to a particular application, need not be used for all applications. Also, not all limitations need be implemented in methods, systems, and apparatuses including one or more concepts described with relation to the provided embodiments. Therefore, the present application properly is to be construed with reference to the claims.

What is claimed:

1. A method for implementing a “breadboard” approach for the study of a biochemical signaling process, pathway, or network, the method comprising:
Separating a naturally occurring multiple-stage biochemical process into a plurality of smaller portions, at least one of which can to a degree of approximation be accurately emulated with a fluidic implementation replica;
Implementing a fluidic implementation replica of at least one of the smaller portions, the replica comprising inputs for reactants, a reaction environment, and provisions for measurement;
Providing the controlled introduction of a plurality of biochemical materials into the replica, and
Making at least one measurement relating to a resulting biochemical reaction within the replica,
Wherein the biochemical reaction are controlled by a computer executing an algorithm, and
Wherein the fluidic implementation replica emulates the at least one the smaller portion of the biochemical signaling process, pathway, or network.

2. The method of claim 1 wherein the replica is configured to provide outlets for removing the reaction products.

3. The method of claim 1 wherein the reaction environment is configured to comprise a membrane

4. The method of claim 1 wherein the replica configured to comprise at least one sensor.

5. The method of claim 1 wherein the replica configured for the measurement is made with an external sensor.

6. The method of claim 1 wherein the replica configured for the measurement is made with an external instrument.

7. The method of claim 1 wherein the replica configured to provide molecular crowding.

8. The method of claim 7 wherein the molecular crowding is implemented as substitute molecular crowding.

9. The method of claim 7 wherein the molecular crowding is implemented as representative molecular crowding

10. The method of claim 1 wherein the replica configured to provide at least one confined reaction environment.

11. The method of claim 1 wherein the replica configured to provide at least one constrained reaction environment.

12. The method of claim 1 wherein a second fluidic implementation replica is used to emulate another smaller portion.

13. The method of claim 12 wherein the replica and the second fluidic implementation replica are configured to be linked fluidically.

14. The method of claim 1 wherein the replica configured to provide the introduction of a competitive species.

15. The method of claim 1 wherein the replica configured to provide the introduction of a drug.

16. The method of claim 1 wherein the replica configured to provide the introduction of an environmental influence.

17. The method of claim 1 wherein the algorithm is a control algorithm.

18. The method of claim 17 wherein the control algorithm comprises a script.

19. The method of claim 1 wherein the algorithm comprises a mathematical model.

20. The method of claim 1 wherein the computer also is executing a mathematical model in communication with the algorithm.
